(19) United States
(12) Patent Application Publication
(10) Pub. No.: US 2023/0364271 A1
(43) Pub. Date: Nov. 16, 2023

(54) METHODS AND COMPOSITIONS FOR INCREASING UPTAKE, INTERNALIZATION, AND/OR RETENTION OF SMALL MOLECULE LIGANDS

(71) Applicant: CORNELL UNIVERSITY, Ithaca, NY (US)

(72) Inventor: Neil H. BANDER, New York, NY (US)

(21) Appl. No.: 18/029,596

(22) PCT Filed: Sep. 28, 2021
(86) PCT No.: PCT/US21/52269

§ 371 Date: Mar. 30, 2023

Related U.S. Application Data
(60) Provisional application No. 63/086,163, filed on Oct. 1, 2020.

(51) Int. Cl. A61K 51/04; A61K 51/10

(52) U.S. Cl. CPC A61K 51/0402; A61K 51/1072; A61K 2121/00

(57) ABSTRACT

The present application relates to methods of treating and imaging cancer. The methods involve providing a first agent comprising a first targeting component coupled to a cancer therapeutic component or an imaging component and providing a second agent comprising a second targeting component alone, wherein the second targeting component increases the uptake, internalization, and/or retention of the first targeting component coupled to a cancer therapeutic component or imaging component. The first and second agents are then administered to a subject having cancer to treat cancer. Also disclosed is a combination therapeutic or a combination imaging system, each comprising the first and second agents.

This application claims the priority benefit of U.S. Provisional Patent Application Ser. No. 63/086,163, filed Oct. 1, 2020, which is hereby incorporated by reference in its entirety.

### FIELD

The present application relates to methods and compositions for increasing uptake, internalization, and/or retention of small molecule ligands.

### BACKGROUND

Combination therapy is a common, accepted treatment approach for virtually all types of cancers and has been the standard therapeutic approach for several decades. The basis for the adoption of combination therapy was the early chemotherapy experience where it was determined that the high mutational rate of cancers allowed rapid development of resistant strains of tumor cells when only a single agent was employed. The goal of combination therapies is to increase efficacy and minimize the development of tumor resistance or escape. This is generally achieved by employing 2 or more anti-cancer agents each of which has a different mechanism of action, making the development of resistant tumor cells more difficult and less likely. The additive or synergistic effects of combining two or more agents can be the difference between successful and unsuccessful treatment of the patient.

Many combination treatment regimens are well known in the oncology field. As an example, MOPP (an acronym for mechlorethamine, vincristine, procarbazine, prednisone) is a curative treatment regimen for Hodgkins' Disease. Several different combination regimens (which all include cisplatin, vinblastine, and bleomycin) are accepted in the treatment of testicular cancer, which is curable in up to 98% of diagnosed cases. In all, more than 300 different combination regimens have been used.

The main drawback to combination therapy is often that it also results in an increase in toxicity. For example, most forms of nonsurgical cancer therapy, such as external irradiation and chemotherapy, are limited in their efficacy because of toxic side effects to normal tissues and cells as well as the limited specificity of these treatment modalities for cancer cells.

This limitation is also of importance when anti-cancer antibodies are used for targeting toxic agents, such as isotopes, drugs, and toxins, to cancer sites, because, as systemic agents, they also circulate to sensitive cellular compartments such as the bone marrow. In acute radiation injury, there is destruction of lymphoid and hematopoietic compartments as a major factor in the development of septicemia and subsequent death. Thus, methods of reducing the toxic effects of cancer therapy while maintaining or even increasing efficacy are in high demand.

Further, although small molecules remain important drugs used in clinics, in numerous cases, their therapeutic impact has reached limitations such as insufficient capability to reach targets, lack of specificity, requirement for high doses leading to toxicity, and major side effects. Pharmaceutical potency of these molecules remains restricted by their poor stability in vivo, their rapid excretion and by their low uptake in cells. Therefore, “delivery” has become a central piece of the therapeutic puzzle and new milestones have been established to validate delivery strategies: (a) lack of toxicity, (b) efficiency at low doses in vivo, (c) easy to handle for therapeutic applications (d) rapid endosomal release and (e) ability to reach the target. Although viral delivery strategies had given much hope for gene and cellular therapies, their clinical application has suffered from side- and toxicity-effects (Glover et al., “Towards Safe, Non-viral Therapeutic Gene Expression in Humans,” Nat. Rev. Genet. 6:299-310 (2005); Whitehead et al., “Knocking Down Barriers: Advances in siRNA Delivery,” Nat Rev Drug Discov. 8:129-138 (2009)). Researchers were mainly focused on the development of non-viral strategies, and different methods have been proposed including lipid, polycationic nanoparticles and peptide-based formulations, but only a few of these technologies have been efficient in vivo and have reached the clinic.

The present application is directed to overcoming these and other deficiencies in the art.

### SUMMARY

A first aspect of the present application relates to a method of treating cancer. The method involves providing a first agent comprising a first targeting component coupled to a cancer therapeutic component and providing a second agent comprising a second targeting component alone, wherein the second agent increases the uptake, internalization, and/or retention of the first targeting component coupled to a cancer therapeutic. The first and second agents are then administered to a subject having cancer to treat cancer.

A second aspect of the present application relates to a combination therapeutic for treating cancer that includes a first agent comprising a first targeting component coupled to a cancer therapeutic and a second agent comprising a second targeting component alone, wherein the second agent increases the uptake, internalization, and/or retention of the first targeting component coupled to a cancer therapeutic.

A third aspect of the present application relates to a method of imaging cancer in a subject. The method involves providing a first agent comprising a first targeting component coupled to an imaging component and providing a second agent comprising a second targeting component alone, wherein the second agent increases the uptake, internalization, and/or retention of the first targeting component coupled to an imaging component. The first and second agents are then administered to a subject having cancer to image cancer.

A fourth aspect of the present application relates to a combination imaging system for imaging cancer. The combination imaging system comprises a first agent comprising a first targeting component coupled to an imaging component and a second agent comprising a second targeting component alone, wherein the second agent increases the uptake, internalization, and/or retention of the first targeting component coupled to an imaging component.

The present application describes a way to achieve improved efficacy of a targeted agent with no increase in, and an opportunity to decrease, its toxicity. The present application proposes the use of two individual targeting agents, rather than one, each targeting the same molecule. In this approach, each of the two targeted agents may have a similar or different biodistribution and/or pharmacokinetics from the other. Having different biodistributions and pharmacokinetics of these respective agents results in differing, non-overlapping toxicities of each of the two respective targeted agents. Alternatively, when the second targeting agent is inherently non-toxic, having different biodistributions and pharmacokinetics of these respective agents is not necessary. When the two targeted agents are combined in a treatment strategy, the result is that both agents converge, simultaneously or sequentially, at the desired target site resulting in an improved treatment effect.

In order to most effectively treat a medical condition, the therapeutic agent should reach the desired target cells. While internalization is often very helpful or even critical, it is not always the case. What is even more important is that it remain at the site for an adequate duration to exert its therapeutic effect. As an example, when one treats a cancer with a ligand-targeted radiopharmaceutical, the treatment effect is commonly compromised by the limited internalization of the ligand-targeted radiopharmaceutical into the tumor cell and further compromised by the rapid efflux of the agent out of the tumor cell where it then diffuses away from the desired site of action. Both the limited uptake/internalization and the rapid efflux limit the tumor's exposure to the therapeutic agent, and, therefore, its beneficial effect is short-circuited.

As described herein, a composition and method has been developed to improve both the internalization/uptake as well as to decrease the efflux of the therapeutic, thereby improving the tumor's exposure (sometimes referred to as “residence time”) to the therapeutic resulting in improved clinical benefit.

In one composition for carrying out this method, the second targeting agent is completely non-toxic such as an antibody. In one embodiment, the antibody would match the species of the treated subject; for example, if the subject is human, the antibody should be a fully human or humanized or at least a de-immunized antibody so that it does not elicit an immune (anti-antibody) response on the part of the treated subject. The antibody comprising the second agent may also be in the form of an antigen binding portion such as antibody fragments well known to those in the art. Typically, the antibody would not be conjugated with any drug or cytotoxin in order to avoid any toxicity resulting from this component.

When the two agents—the first targeting agent coupled with its therapeutic component and the second targeting agent—are administered, the latter targeting agent improves the internalization, uptake, and retention of the first agent within the treated cells. This improves the therapeutic effect of the first targeting agent coupled with its therapeutic component. Further, it allows the administration of a higher amount of the first targeting agent-therapeutic providing the opportunity to increase potency. Alternatively, because there is greater effect from the administered first targeting agent-therapeutic component, when administered along with the second targeting agent, the dose of the first targeting agent-therapeutic can be lowered while still achieving the same level of clinical activity yet decreasing toxicity that the first targeting agent-therapeutic would otherwise cause when given at its standard dose.

Most commonly, the two targeting moieties of the composition of the present application will consist of a small molecule ligand and an antibody (or an antigen binding portion thereof). Most commonly, the two targeting moieties of this composition will bind to different, non-overlapping (i.e., non-competitive) sites of the same molecular target as demonstrated in the examples below. However, once a first targeting agent has been identified, one of skill in the art can readily screen for a second targeting agent that results in enhanced uptake, internalization, and retention when combined with the first. Further, the screening can include the option that the 2 agents may be of different physical nature than the small molecule ligand and antibody and/or where the 2 agents target different molecular targets on the same cell.

### BRIEF DESCRIPTION OF THE DRAWINGS

FIGS. 1A-1B show that the small molecule ligand is retained poorly within tumor cell relative to an antibody in an in vitro assay using 2 human prostate cancer cell lines. In this assay, the small molecule and the antibody are given individually. While approximately 80% of the antibody is retained over the 6 day period, the small molecule ligand is rapidly effluxed out of the cells.

FIG. 2 shows that the J591 antibody improves the amount of PSMA-617-Lu¹⁷⁷ internalization after a 3 hour incubation in vitro (at time 0) and thereafter improves the absolute retention of the small molecule ligand over the 48 hour period of measurement. This effect is shown in 2 different human prostate cancer cell lines in vitro.

FIG. 3 shows that cells that were exposed to both J591 and ACUPA-Cy3 internalized and retained approximately 2-fold the ACUPA dye as those cells that did not have J591 present. This measurement of the area under the curve was made by confocal microscopy and computer digitized measurements.

FIG. 4 shows that co-administration of cold J591 (targeting PSMA) at approximately 1 to 2x molar amount relative to PSMA-617-Lu¹⁷⁷ increased uptake of PSMA-617-Lu¹⁷⁷ in an animal model of prostate cancer. Herceptin served as a negative control antibody and showed no effect.

FIG. 5 shows the early impact of J591 anti-PSMA antibody on PSMA ligand (PSMA I&T-Lu¹⁷⁷) internalization/retention in vivo in an animal model. After co-injection of unlabeled antibody plus PSMA I&T-Lu^{177,} mice were euthanized 2 hours later. Their tumors were harvested, weighed, and counted. Herceptin served as a negative control antibody. This experiment demonstrated that the anti-PSMA antibody increased the uptake and retention of the small molecule ligand by 36% and showing the effect of the combination occurs rapidly.

### DETAILED DESCRIPTION

A first aspect of the present application is directed to a method of treating cancer that involves providing a first agent comprising a first targeting component coupled to a cancer therapeutic component and providing a second agent comprising a second targeting component alone, wherein the second agent increases the uptake, internalization, and/or retention of the first targeting component coupled to a cancer therapeutic. The first and second agents are then administered, to a subject having cancer, to treat cancer.

As used herein, the term “subject” is intended to include human and non-human animals. Non-human animals include all vertebrates, e.g., mammals and non-mammals, such as non-human primates, sheep, dog, cow, chickens, amphibians, reptiles, etc.

As used herein, the term “treat” refers to the application or administration of the first and second agents of the application to a subject, e.g., a patient. The treatment can be to cure, heal, alleviate, relieve, alter, remedy, ameliorate, palliate, improve or affect the cancer, the symptoms of the cancer or the predisposition toward the cancer.

As used herein, the term “cancer” includes all types of cancerous growths or oncogenic processes, metastatic tissues or malignantly transformed cells, tissues, or organs, irrespective of histopathologic type or stage of invasiveness.

As used herein, the term “uptake” refers to the intial entry of the first targeting compound coupled to a cancer therapeutic into a cell.

As used herein, the term “internalization” refers to the delivery of the first targeting compound coupled to a cancer therapeutic into a cell, e.g., the cytosol.

As used herein, the term “retention” refers to the length of time that the first targeting compound coupled to a cancer therapeutic remains inside a cell.

As used herein, the term “biodistribution” refers to the organs and tissues to which a drug distributes in the body.

As used herein, the term “pharmacokinetics” refers to how long a drug stays in the body.

In certain embodiments, the cancer is prostate cancer, neuroendocrine cancer, breast cancer, or non-Hodgkin's lymphoma. In some embodiments, the cancer is a primary tumor, while in other embodiments, the cancer is a secondary or metastatic tumor.

As used herein, the “targeting component” is a component that is able to bind to or otherwise associate with a molecular target, for example, a membrane component, a cell surface receptor, prostate specific membrane antigen (PSMA, which is also known as folate hydrolase 1, glutamate carboxypeptidase II, and NAALADase), or the like. A first and second agent comprising the targeting component may become co-localized or converge at a particular targeted site, for instance, a tumor, a disease site, a tissue, an organ, a type of cell, etc. As such, the first and second agent may be “target-specific.” In some cases, the therapeutic component that is coupled to the first targeting component may exert its anti-cancer effect without the need for release from the first targeting component. In other cases, the therapeutic component may be released from the first agent and allowed to interact locally with the particular targeting site.

For example, contemplated targeting components may include a nucleic acid, peptide, polypeptide, protein, glycoprotein, carbohydrate, or lipid. A targeting component may be a naturally occurring or synthetic ligand for a cell surface receptor, e.g., a growth factor, hormone, LDL, transferrin, etc. A targeting component can be an antibody, which term is intended to include antibody fragments, characteristic portions of antibodies, single chain targeting moieties which can be identified, for example, using procedures such as phage display. Targeting components may also be a targeting peptide, targeting peptidomimetic, or a small molecule, whether naturally-occurring or artificially created (e.g., via chemical synthesis).

In one embodiment, the first targeting component is selected from the group consisting of a protein, a peptide, and a small molecule, and the second target component is an antibody or binding fragment thereof.

Antibodies against molecular targets on tumors are known. For example, antibodies and antibody fragments which specifically bind markers produced by or associated with tumors have been disclosed, inter alia, in U.S. Pat. No. 3,927,193 to Hansen, and U.S. Pat. Nos. 4,331,647, 4,348,376, 4,361,544, 4,468,457, 4,444,744, 4,818,709 and 4,624,846 to Goldenberg, the contents of all of which are incorporated herein by reference in their entirety. In particular, antibodies against an antigen, e.g., a gastrointestinal, lung, breast, prostate, ovarian, testicular, brain or lymphatic tumor, a sarcoma or a melanoma, are advantageously used. Antibodies to cancer-related antigens are well known to those in the art.

The antibodies of the present application may exist in a variety of forms including, for example, polyclonal antibodies, monoclonal antibodies, intracellular antibodies (“intrabodies”), antibody fragments (e.g. Fv, Fab and F(ab)2), half-antibodies, hybrid derivatives, as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), each of which is hereby incorporated by reference in its entirety).

Antibodies of the present application may also be synthetic antibodies. A synthetic antibody is an antibody which is generated using recombinant DNA technology, such as, for example, an antibody expressed by a bacteriophage. Alternatively, the synthetic antibody is generated by the synthesis of a DNA molecule encoding and expressing the antibody of the present application or the synthesis of an amino acid sequence specifying the antibody, where the DNA or amino acid sequence has been obtained using synthetic DNA or amino acid sequence technology which is available and well known in the art.

Methods for monoclonal antibody production may be carried out using the techniques described herein or are well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest either in vivo or in vitro.

Alternatively monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the present application can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

In addition to whole antibodies, the present application encompasses binding portions of such antibodies. Such binding portions include the monovalent Fab fragments, Fv fragments (e.g., single-chain antibody, scFv), and single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antibody mimics are also suitable for use in accordance with the present application. A number of antibody mimics are known in the art including, without limitation, those known as monobodies, which are derived from the tenth human fibronectin type III domain (&¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

The peptides used in conjunction with the present application can be obtained by known isolation and purification protocols from natural sources, can be synthesized by standard solid or solution phase peptide synthesis methods according to the known peptide sequence of the peptide, or can be obtained from commercially available preparations. Included herein are peptides that exhibit the biological binding properties of the native peptide and retain the specific binding characteristics of the native peptide. Derivatives, analogs, and antigen binding portions of the peptide, as used herein, include modifications in the composition, identity, and derivitization of the individual amino acids of the peptide provided that the peptide retains the specific binding properties of the native peptide. Examples of such modifications would include modification of any of the amino acids to include the D-stereoisomer, substitution in the aromatic side chain of an aromatic amino acid, derivitization of the amino or carboxyl groups in the side chains of an amino acid containing such a group in a side chain, substitutions in the amino or carboxy terminus of the peptide, linkage of the peptide to a second peptide or biologically active moiety, and cyclization of the peptide (G. Van Binst and D. Tourwe, “Backbone Modifications in Somatostatin Analogues: Relation Between Conformation and Activity,” Peptide Research 5:8-13 (1992), which is hereby incorporated by reference in its entirety).

In one embodiment, the first and second targeting components target the same molecular target. For example, the first and second targeting components may bind to the same receptor (e.g. PSMA) expressed by the same cell type.

In another embodiment, the first and second targeting components target different molecular targets on the same cell type. For example, the first and second targeting components may bind to different receptors (e.g. HER1 and HER2) expressed on the same cell type.

As used herein, the “cancer therapeutic component” is an agent, or combination of agents, that treats a cell, tissue, or subject having a condition requiring therapy, when contacted with the cell, tissue or subject. The cancer therapeutic component may be, for example, a therapeutic radionuclide, chemotherapeutic agent, cytotoxin, hormone, hormone antagonist, receptor antagonist, enzyme or proenzyme activated by another agent, biologic, autocrine or cytokine. Toxins also can be used in the methods of the present application. Other therapeutic agents useful in the present application include anti-DNA, anti-RNA, radiolabeled oligonucleotides, such as anti-sense oligodeoxy ribonucleotides, anti-protein and anti-chromatin cytotoxic or antimicrobial agents. Other therapeutic agents are known to those skilled in the art, and the use of such other therapeutic agents in accordance with the present application is specifically contemplated.

In one embodiment, the cancer therapeutic component is selected from the group consisting of a radionuclide and a chemotherapeutic agent.

In one embodiment, the cancer therapeutic component is a radionuclide selected from the group consisting of ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁸⁸Rd, ¹⁶⁶Dy, ¹⁶⁶ho, ¹⁴⁹pm, ¹⁵¹Pm, ¹⁵³Sm, ¹⁵⁹Gd, ¹⁷²Tm, ¹⁶⁹yb, ¹⁷⁵Yb, ¹⁷⁷Lu, ¹⁰⁵ Rh, ¹¹¹Ag, ¹³¹I, ¹⁷⁷mSn, ²²⁵Ac, ²²⁷Th, ²¹¹At, and combinations thereof.

Procedures for labeling agents with radioactive isotopes are generally known in the art. For example, there are a wide range of moieties which can serve as chelating ligands and which can be derivatized to the first targeting component of the application. For instance, the chelating ligand can be a derivative of 1,4,7,10-tetraazacyclododecanetetraacetic acid (DOTA), ethylenediaminetetraacetic acid (EDTA), diethylenetriaminepentaacetic acid (DTPA), and 1-p-Isothiocyanato-benzyl-methyl-diethylenetriaminepentaacetic acid (ITC-MX). These chelators typically have groups on the side chain by which the chelator can be used for attachment to the first targeting component of the present application. Such groups include, e.g., benzylisothiocyanate, by which the DOTA, DTPA, or EDTA can be coupled to, e.g., an amine group of the targeting component. Procedures for iodinating biological agents, such as antibodies, binding portions thereof, probes, or ligands, are described by Hunter and Greenwood, “Preparation of Iodine-131 Labelled Human Growth Hormone of High Specific Activity,” Nature 144:496-496 (1962), David et al., “Protein Iodination With Solid State Lactoperoxidase,” Biochemistry 13:1014-1021 (1974), and U.S. Pat. Nos. 3,867,517 to Ling and 4,376,110 to David, which are hereby incorporated by reference in their entirety. Other procedures for iodinating biological agents are described by Greenwood et al., “The Preparation of I-131-Labelled Human Growth Hormone of High Specific Radioactivity,” Biochem. J. 89:114-123 (1963); Marchalonis, “An Enzymic Method for the Trace Iodination of Immunoglobulins and Other Proteins,” Biochem. J. 113:299-305 (1969); and Morrison et al., “Use of Lactoperoxidase Catalyzed Iodination in Immunochemical Studies,” Immunochemistry 8:289-297 (1971), which are hereby incorporated by reference in their entirety. Procedures for ⁹⁹m Tc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference in their entirety. Procedures suitable for 111 In-labeling biological agents are described by Hnatowich et al., “The Preparation of DTPA-coupled Antibodies Radiolabeled With Metallic Radionuclides: an Improved Method,” J. Immul. Methods 65:147-157 (1983), Hnatowich et al., “Coupling Antibody With DTPA—an Alternative to the Cyclic Anhydride,” Int. J. Applied Radiation 35:554-557 (1984), and Buckley et al., “An Efficient Method For Labelling Antibodies With 111In,” F.E.B.S. 166:202-204 (1984), which are hereby incorporated by reference in their entirety.

In another embodiment, the cancer therapeutic component is a chemotherapeutic agent selected from the group consisting of busulfan, cisplatin, carboplatin, chlorambucil, cyclophosphamide, ifosfamide, dacarbazine (DTIC), mechlorethamine (nitrogen mustard), melphalan carmustine (BCNU), lomustine (CCNU), 5-fluorouracil (5-FU), capecitabine, methotrexate, gemcitabine, cytarabine (ara-C), fludarabine, dactinomycin, daunorubicin, doxorubicin (Adriamycin), idarubicin, mitoxantrone, paclitaxel, docetaxel, cabazitaxel, etoposide (VP-16), vinblastine, vincristine, vinorelbine, prednisone, dexamethasone, tamoxifen, fulvestrant, anastrozole, letrozole, megestrol acetate, bicalutamide, flutamide, leuprolide, goserelin, L-asparaginase, tretinoin, maytansines, auristatins, pyrrolobenzodiazepines, duocarmycins, and combinations thereof.

Procedures for conjugating biological agents with chemotherapeutic agents are well known in the art. Most of the chemotherapeutic agents currently in use in treating cancer possess functional groups that are amenable to chemical crosslinking directly with an amine or carboxyl group of the first targeting component of the present application. For example, free amino groups are available on methotrexate, doxorubicin, daunorubicin, cytosinarabinoside, cisplatin, vindesine, mitomycin, and bleomycin while free carboxylic acid groups are available on methotrexate, melphalan, and chlorambucil. These functional groups, that is free amino and carboxylic acids, are targets for a variety of homo-bifunctional and hetero-bifunctional chemical crosslinking agents which can crosslink these drugs directly to a free amino group of the first targeting component. Specific procedures for conjugating targeting components with chemotherapeutic agents have been described and are known in the art. By way of example, conjugation of chlorambucil with antibodies is described by Flechner, “The Cure and Concomitant Immunization of Mice Bearing Ehrlich Ascites Tumors by Treatment With an Antibody--Alkylating Agent Complex,” European Journal of Cancer 9:741-745 (1973); Ghose et al., “Immunochemotherapy of Cancer with Chlorambucil-Carrying Antibody,” British Medical Journal 3:495-499 (1972); and Szekerke et al., “The Use of Macromolecules as Carriers of Cytotoxic Groups (part II) Nitrogen Mustard—Protein Complexes,” Neoplasma 19:211-215 (1972), which are hereby incorporated by reference in their entirety. Procedures for conjugating daunomycin and adriamycin to antibodies are described by Hurwitz et al., “The Covalent Binding of Daunomycin and Adriamycin to Antibodies, With Retention of Both Drug and Antibody Activities,” Cancer Research 35:1175-1181 (1975) and Arnon et al. Cancer Surveys 1:429-449 (1982), which are hereby incorporated by reference in their entirety. Coupling procedures are also described in EP 86309516.2, which is hereby incorporated by reference in its entirety.

It will be appreciated that the exact dosage of the first and second agents of the application is chosen by the individual physician in view of the patient to be treated. In general, dosage and administration are adjusted to provide an effective amount of the agent to the patient being treated. As used herein, the “effective amount” of an agent refers to the amount necessary to elicit the desired biological response. As will be appreciated by those of ordinary skill in this art, the effective amount of agent may vary depending on such factors as the desired biological endpoint, the drug to be delivered, the target tissue, the route of administration, etc. For example, the effective amount of agent containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

In general, doses can range from about 25% to about 100% of the MTD of the targeted agent when given as a single agent. Based upon the composition, the dose can be delivered once, continuously, such as by continuous pump, or at periodic intervals. Dosage may be adjusted appropriately to achieve desired drug levels, locally, or systemically. In the event that the response in a subject is insufficient at such doses, even higher doses (or effective higher doses by a different, more localized delivery route) may be employed to the extent that patient tolerance permits. Continuous IV dosing over, for example, 24 hours or multiple doses per day also are contemplated to achieve appropriate systemic levels of compounds.

In one embodiment, the cancer therapeutic component has a maximum tolerated dose, and the maximum tolerated dose of the cancer therapeutic component is administered to the subject.

In an alternative embodiment, less than the maximum tolerated dose of the cancer therapeutic component is administered to the subject. When the two agents of the present application are combined in a treatment strategy, the result is that both agents converge (simultaneously or sequentially) at the desired target site thereby providing an enhanced treatment effect and, because the therapeutic component of the first agent is administered at less than its MTD, lower toxicity is experienced by the subject.

In one embodiment, the first agent is a small molecule conjugated to a radionuclide and is administered in a 2-week cycle at a total dose of about 300 to 900 mCi (11.0-33.3 GBq), such as a dose of 300, 325, 350, 375, 400, 425, 450, 475, 500, 600, 700, 800 or 900 mCi total in a 2 week cycle.

In practicing the methods of the present application, the administering step is carried out to treat cancer in a subject. In one embodiment, a subject having cancer is selected prior to the administering step. Such administration can be carried out systemically or via direct or local administration to the tumor site. By way of example, suitable modes of systemic administration include, without limitation, orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent (e.g., an antibody or an inhibitory nucleic acid molecule) and the disease to be treated.

The agents of the present application may be orally administered, for example, with an inert diluent, or with an assimilable edible carrier, or it may be enclosed in hard or soft shell capsules, or it may be compressed into tablets, or they may be incorporated directly with the food of the diet. Agents of the present application may also be administered in a time release manner incorporated within such devices as time-release capsules or nanotubes. Such devices afford flexibility relative to time and dosage. For oral therapeutic administration, the agents of the present application may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present application in such therapeutically useful compositions is such that a suitable dosage will be obtained.

When the agents of the present application are administered parenterally, solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present application systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing, and/or dispersing agents.

Intraperitoneal or intrathecal administration of the agents of the present application can also be achieved using infusion pump devices. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

According to one embodiment, the cancer is prostate cancer.

In another embodiment of this aspect of the present application, when the cancer is prostate cancer, the first and second targeting components target the PSMA receptor.

As used herein, “PSMA” or “prostate-specific membrane antigen” protein refers to mammalian PSMA, preferably human PSMA protein. The long transcript of PSMA encodes a protein product of about 100-120 kDa molecular weight characterized as a type II transmembrane receptor having sequence homology with the transferrin receptor and having NAALADase activity (Carter et al., “Prostate-Specific Membrane Antigen is a Hydrolase With Substrate and Pharmacologic Characteristics of a Neuropeptidase,” Proc. Natl. Acad. Sci. USA 93:749-753 (1996), which is hereby incorporated by reference in its entirety).

In an alternative embodiment, the first targeting component is a PSMA receptor binding peptide or PSMA receptor inhibitor and the second targeting component is a PSMA receptor antibody or an antigen binding portion thereof.

A PSMA receptor antibody is an antibody that interacts with (e.g., binds to) PSMA, preferably human PSMA protein. Preferably, the PSMA receptor antibody interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866, which is hereby incorporated by reference in its entirety). PSMA receptor antibodies are known in the art (Goldsmith et al., “Targeted Radionuclide Therapy for Prostate Cancer,” in Therapeutic Nuclear Medicine 617-628 (R. Baum ed. 2014), which is hereby incorporated by reference in its entirety). Exemplary PSMA receptor antibodies include, but are not limited to, J591, J415, J533, and E99.

The PSMA receptor inhibitor may include any lipids, carbohydrates, polynucleotides, peptides, polypeptides, or any other biologic, organic or inorganic molecules which inhibit the function of the PSMA receptor. Exemplary PSMA receptor inhibitor are known in the art include, but are not limited to, PSMA 617, PSMA I&T, DCFBC, DCFPyL, glutamate-urea-lysine analogs, phosphoramidate analogs, and 2-(phosphinylmethyl) pentanedioic acid analogs (Lutje et al., “PSMA Ligands for Radionuclide Imaging and Therapy of Prostate Cancer: Clinical Status,” Theranostics 5(12):1388-1401 (2015); Haberkorn et al., “New Strategies in Prostate Cancer: Prostate-Specific Membrane Antigen (PSMA) Ligands for Diagnosis and Therapy,” Clin. Cancer Res. 22(1):9-15 (2016), which are hereby incorporated by reference in their entirety).

In one embodiment, the PSMA receptor antibody is selected from the group consisting of J591, J415, J533, and E99, while the first targeting component is a peptide selected from the group consisting of PSMA 617, PSMA I&T, DCFBC, DCFPyL, glutamate-urea-lysine analogs, phosphoramidate analogs, 2-(phosphinylmethyl) pentanedioic acid analogs, and other PSMA ligands/inhibitors.

In one embodiment, the first agent is PSMA 617-¹⁷⁷Lu or PSMA I&T-¹⁷⁷Lu and the second agent is J591.

In another embodiment of the present application, the cancer is a neuroendocrine cancer. Neuroendocrine cancers include, but are not limited to, carcinoid tumors, gastrinoma, insulinoma, glucagonoma, VIPoma, somatostatinoma, thyroid carcinoma, Merkel cell carcinoma of the skin, tumor of the anterior pituitary, medullary carcinoma, parathyroid tumor, thymus and mediastinal carcinoid tumor, pulmonary neuroendocrine tumor, adrenomedullary tumor, pheochromocytoma, Schwannoma, paraganglioma, and neuroblastoma.

In accordance with this aspect of the present application, in one embodiment, the first and second targeting components target the somatostatin receptor.

At least five somatostatin receptors subtypes have been characterized and tumors can express various receptor subtypes (Shaer et al., “Somatostatin Receptor Subtypes sst1, sst2, sst3 and sst5 Expression in Human Pituitary, Gastroentero-Pancreatic and Mammary tumors: Comparison of mRNA Analysis With Receptor Autoradiography,” Int. J. Cancer 70:530-537 (1997), which is hereby incorporated by reference in its entirety). Naturally occurring somatostatin and its analogs exhibit differential binding to these receptor subtypes, allowing precise targeting of a peptide analog to specific diseased tissues.

In accordance with this aspect of the application, the first and second targeting components have at least one biological activity of native somatostatin; preferably, this activity is the ability to specifically bind to a somatostatin receptor on a somatostatin receptor-bearing cell. Many such analogs having biological activity are known and have been described, for example, in U.S. Pat. No. 5,770,687 to Hornik et al.; U.S. Pat. No. 5,708,135 to Coy et al.; U.S. Pat. No. 5,750,499 to Hoeger et al; U.S. Pat. No. 5,620,675 to McBride et al.; U.S. Pat. No. 5,633,263 to Coy et al; U.S. Pat. No. 5,597,894 to Coy et al; U.S. Pat. No. 5,073,541 to Taylor et al; U.S. Pat. No. 4,904,642 to Coy et al; U.S. Pat. No. 6,017,509 to Dean; WO 98/47524 to Hoffman et al.; and U.S. Pat. No. 5,411,943 to Bogden, each of which is hereby incorporated by reference in its entirety.

In one embodiment, the first and second targeting components target the somatostatin receptor-2.

In another embodiment of the present application, the cancer is breast cancer.

In accordance with this embodiment of the present application, when the cancer is breast cancer, the first and second targeting components target the HER receptor family.

First and second agents, as well as targeting and therapeutic components, are described above.

In another embodiment of the present application the cancer is non-Hodgkin's Lymphoma.

In accordance with this embodiment, when the cancer is non-Hodgkin's lymphoma, the first and second targeting components target CD20.

Another aspect of the present application relates to a combination therapeutic for treating cancer that includes a first agent comprising a first targeting component coupled to a cancer therapeutic and a second agent comprising a second targeting component alone. The second agent increases the uptake, internalization, and/or retention of the first targeting component coupled to a cancer therapeutic.

First and second agents, as well as targeting and therapeutic components, are described above.

Pharmaceutical compositions containing agents for use in the methods of the present application can include a pharmaceutically acceptable carrier as described infra, one or more active agents, and a suitable delivery vehicle. Suitable delivery vehicles include, but are not limited to, viruses, bacteria, biodegradable microspheres, microparticles, nanoparticles, liposomes, collagen minipellets, and cochleates.

In one embodiment of the present application, the pharmaceutical composition or formulation containing an inhibitory nucleic acid molecule (e.g., siRNA molecule) is encapsulated in a lipid formulation to form a nucleic acid-lipid particle as described in Semple et al., “Rational Design of Cationic Lipids for siRNA Delivery,” Nature Biotech. 28:172-176 (2010), WO2011/034798 to Bumcrot et al., WO2009/111658 to Bumcrot et al., and WO2010/105209 to Bumcrot et al., which are hereby incorporated by reference in their entirety.

In another embodiment of the present application, the delivery vehicle is a nanoparticle. A variety of nanoparticle delivery vehicles are known in the art and are suitable for delivery of an inhibitor of the application (see e.g., van Vlerken et al., “Multi-functional Polymeric Nanoparticles for Tumour-Targeted Drug Delivery,” Expert Opin. Drug Deliv. 3(2):205-216 (2006), which is hereby incorporated by reference in its entirety). Suitable nanoparticles include, without limitation, poly(beta-amino esters) (Sawicki et al., “Nanoparticle Delivery of Suicide DNA for Epithelial Ovarian Cancer Cell Therapy,” Adv. Exp. Med. Biol. 622:209-219 (2008), which is hereby incorporated by reference in its entirety), polyethylenimine-alt-poly(ethylene glycol) copolymers (Park et al., “Degradable Polyethylenimine-alt-Poly(ethylene glycol) Copolymers As Novel Gene Carriers,” J. Control Release 105(3):367-80 (2005) and Park et al., “Intratumoral Administration of Anti-KITENIN shRNA-Loaded PEI-alt-PEG Nanoparticles Suppressed Colon Carcinoma Established Subcutaneously in Mice,” J Nanosci. Nanotechnology 10(5):3280-3 (2010), which are hereby incorporated by reference in their entirety), and liposome-entrapped siRNA nanoparticles (Kenny et al., “Novel Multifunctional Nanoparticle Mediates siRNA Tumor Delivery, Visualization and Therapeutic Tumor Reduction In Vivo,” J. Control Release 149(2): 111-116 (2011), which is hereby incorporated by reference in its entirety). Other nanoparticle delivery vehicles suitable for use in the present application include microcapsule nanotube devices disclosed in U.S. Patent Publication No. 2010/0215724 to Prakash et al., which is hereby incorporated by reference in its entirety.

In another embodiment of the present application, the pharmaceutical composition is contained in a liposome delivery vehicle. The term “liposome” means a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Several advantages of liposomes include: their biocompatibility and biodegradability, incorporation of a wide range of water and lipid soluble drugs; and they afford protection to encapsulated drugs from metabolism and degradation. Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Methods for preparing liposomes for use in the present application include those disclosed in Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda, and U.S. Pat. No. 5,059,421 to Loughrey et al., which are hereby incorporated by reference in their entirety.

In another embodiment of the present application, the delivery vehicle is a viral vector. Viral vectors are particularly suitable for the delivery of inhibitory nucleic acid molecules, such as siRNA or shRNA molecules, but can also be used to deliver molecules encoding an anti-integrin antibody. Suitable gene therapy vectors include, without limitation, adenoviral vectors, adeno-associated viral vectors, retroviral vectors, lentiviral vectors, and herpes viral vectors.

Adenoviral viral vector delivery vehicles can be readily prepared and utilized as described in Berkner, “Development of Adenovirus Vectors for the Expression of Heterologous Genes,” Biotechniques 6:616-627 (1988), Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral delivery vehicles can be constructed and used to deliver an inhibitory nucleic acid molecule of the present application to cells as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l. Acad. Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver a nucleic acid molecule to a target cell. One such type of retroviral vector is disclosed in U.S. Pat. No. 5,849,586 to Kriegler et al., which is hereby incorporated by reference. Other nucleic acid delivery vehicles suitable for use in the present application include those disclosed in U.S. Patent Publication No. 20070219118 to Lu et al., which is hereby incorporated by reference in its entirety.

Regardless of the type of infective transformation system employed, it should be targeted for delivery of the nucleic acid to the desired cell type. For example, for delivery into a cluster of cells (e.g., cancer cells) a high titer of the infective transformation system can be injected directly within the site of those cells so as to enhance the likelihood of cell infection. The infected cells will then express the inhibitory nucleic acid molecule targeting the inhibition of integrin expression. The expression system can further contain a promoter to control or regulate the strength and specificity of expression of the nucleic acid molecule in the target tissue or cell.

Effective doses of the compositions of the present application, for the treatment of a metastatic disease vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

A further aspect of the present application relates to a method of imaging cancer in a subject. The method involves providing a first agent comprising a first targeting component coupled to an imaging component and providing a second agent comprising a second targeting component alone. The second agent increases the uptake, internalization, and/or retention of the first targeting component coupled to an imaging component. The first and second agents are then administered to a subject having cancer to image cancer.

First and second agents, as well as targeting components, are described above.

As used herein, an “imaging component” an agent utilized to detect cancerous tissues (particularly the vascular endothelial cells therein) in vivo. This is achieved by coupling the imaging component to the first targeting component, administering the first and second agents to a subject having cancer, and then imaging the subject.

Examples of imaging components in accordance with the present application are radiolabels such as Ga⁶⁸, F¹⁸, Cu⁶⁷, ¹³¹I, ¹¹¹In, ¹²³I, ⁹⁹mTc, ³²P, ¹²⁵I, ³H, ¹⁴C and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. These isotopes and transrectal detector probes, when used in combination, are especially useful in detecting prostatic fossa recurrences and pelvic nodal disease. The first agent can be labeled with such reagents using techniques known in the art.

In the case of a radiolabeled agent, the biological agent is administered to the patient, is localized to the tumor bearing the antigen with which the biological agent reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference in its entirety. Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

As used herein, imaging can include any one or more of: planar radionuclide imaging, positron emission tomography (PET), echo-planar imaging (EPI), single photon emission computed tomography (SPECT), sonographic imaging (e.g., radiation-free, contrast-specific, high frequency, two-dimensional), magnetic resonance imaging (MRI, also referred to as magnetic resonance tomography or MRT), X-ray, computed tomographic (CT) scans, fluorescence imaging, near-infrared imaging and other medically useful or adaptable imaging techniques.

Fluorophore and chromophore labeled agents can be prepared from standard moieties known in the art. Since proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer, Science 162:526 (1968) and Brand, L. et al., Annual Review of Biochemistry 41:843-868 (1972), which are hereby incorporated by reference in their entirety. The first agent can be labeled with fluorescent chromophore groups by conventional procedures such as those disclosed in U.S. Pat. Nos. 3,940,475, 4,289,747, and 4,376,110, which are hereby incorporated by reference in their entirety.

One group of fluorescers having a number of the desirable properties described above are the xanthene dyes, which include the fluoresceins derived from 3,6-dihydroxy-9-henylxanthhydrol and resamines and rhodamines derived from 3,6-diamino-9-phenylxanthydrol and lissanime rhodamine B. The rhodamine and fluorescein derivatives of 9-o-carboxyphenylxanthhydrol have a 9-o-carboxyphenyl group. Fluorescein compounds having reactive coupling groups such as amino and isothiocyanate groups such as fluorescein isothiocyanate and fluorescamine are readily available. Another group of fluorescent compounds are the naphthylamines, having an amino group in the α or β position.

A fourth aspect of the present application relates to a combination imaging system for imaging cancer. The combination imaging system comprises a first agent comprising a first targeting component coupled to an imaging component and a second agent comprising a second targeting component alone. The second agent increases the uptake, internalization, and/or retention of the first targeting component coupled to an imaging component. This aspect of the present application utilizes the components described above with reference to the method of imaging set forth above.

### EXAMPLES

The following examples are provided to illustrate embodiments of the present application, but they are by no means intended to limit its scope.

Example 1

### Small Molecule Ligand is Retained Poorly Within Tumor Cell Relative to Antibody In Vitro

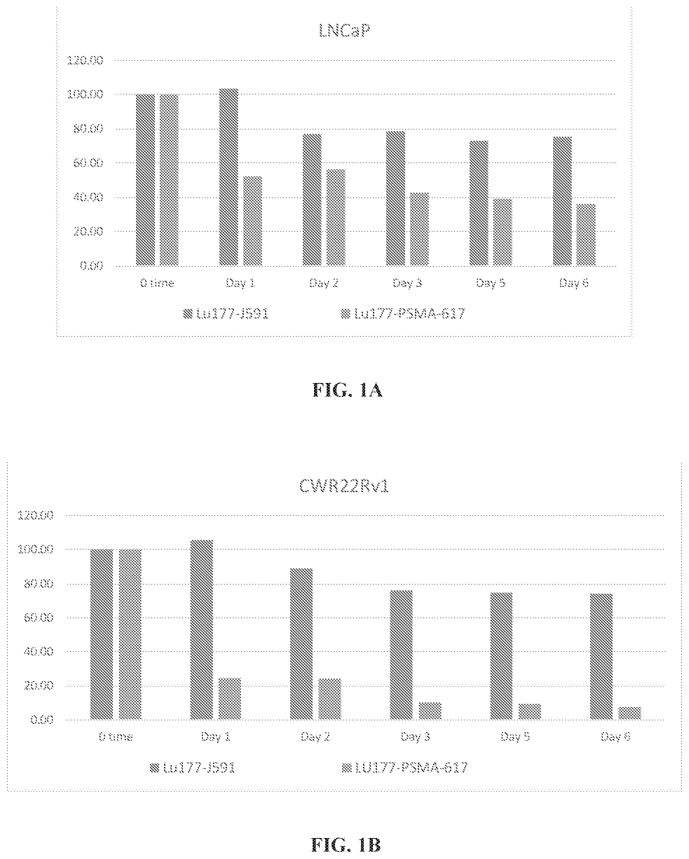

LNCaP or CWR22Rv1 cells were plated into 12-well plates overnight. Lu¹⁷⁷-J591 (0.5 μci/ml/well) or Lu¹⁷⁷-PSMA-617 (0.5 μci/ml/well) was then added, and the cells were incubated for 6 hours at 37° C. to allow internalization. Duplicate wells were used for each time point. After 6 hours of incubation, the cells were washed once with RPMI-10% FBS. 0.6 ml of 1% TritonX-100 was added per well for 2 wells as 0 time point, and 1.5 ml of RPMI-10% FBS was added to the remaining wells. The cells were incubated at 37° C. for 1 to 6 days. On day 1, 2, 3, 5, and 6, the medium was removed and 0.6 ml of 1% TritonX-100 was added to each well. The lysate was collected from each well and all samples were counted at day 6. Results are shown in Table 1 below (in % of time 0 counts) and FIGS. 1A-1B.

TABLE 1

| | | | CWR22Rv1 | |
|---|---|---|---|---|
| | LNCaP | Lu¹⁷⁷- | | LU¹⁷⁷- |
| | Lu¹⁷⁷-J591 | PSMA-617 | Lu¹⁷⁷-J591 | PSMA-617 |
| 0 time | 100.00 | 100.00 | 100.00 | 100.00 |
| Day 1 | 103.39 | 52.21 | 105.37 | 24.37 |
| Day 2 | 76.74 | 56.06 | 88.74 | 23.99 |
| Day 3 | 78.65 | 42.80 | 75.92 | 10.28 |
| Day 5 | 73.14 | 39.12 | 74.47 | 9.39 |
| Day 6 | 75.13 | 35.82 | 74.12 | 7.64 |

The above findings, in 2 human prostate cancer cell lines, suggests that, in contrast to the PSMA antibody, the small molecule ligand, PSMA 617-Lu^{177,} is rapidly effluxed back out of the cell soon after internalization. This is consistent with the natural physiology of many ligand-receptor pairs (e.g., transferrin-transferrin receptor) where the ligand is initially internalized only to be recycled back out of the cell. In contrast, the antibody, which is not a natural ligand for the receptor, and which has a high avidity binding through its dimeric structure, is retained far longer. The practical importance of this is that whatever payloads are transported by these different entities are retained in the targeted tumor cell much longer when delivered by antibody than by small molecule ligand.

### Example 2

### J591 Improves Internalization and Retention of SML

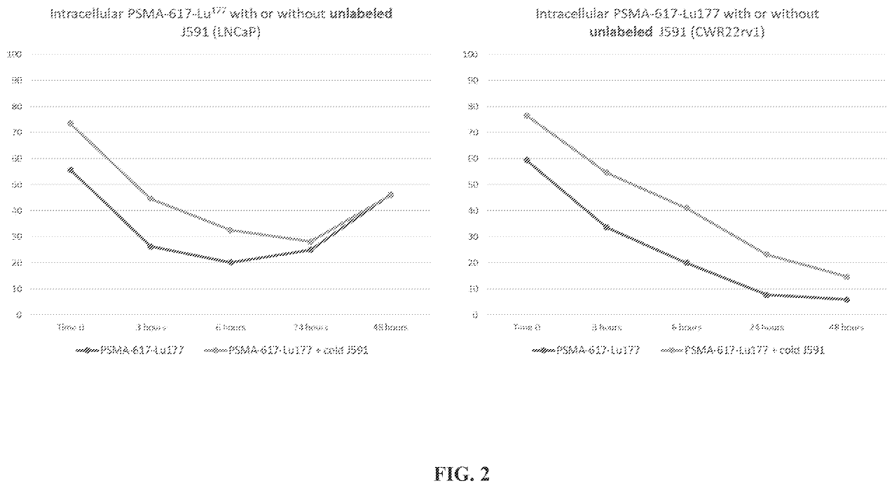

In 2 human prostate cancer cell lines, LNCaP (androgen responsive) and CWR22Rv1 (castrate resistant), the effect of “cold” (unlabeled) J591 on the internalization and retention of PSMA-617-¹⁷⁷Lu was measured. Two conditions were studied: cells were incubated with PSMA-617-¹⁷⁷Lu with or without J591, for 3 hours at which point the PSMA-617 and J591 are removed and the cells washed with PBS. This point represents time 0. The cell-associated counts in the well at time 0 represent 100%. At 0, 3, 6, 24 and 48 hour time points, media is removed and counted (representing externalized counts) and the cell surface is stripped of any counts using a low pH (2.8) incubation. The cells are then harvested from the wells, and the intracellular counts are determined in a gamma counter and plotted as a % of the counts at time 0. The data shows that, at time 0, the cells that got both PSMA-617-Lu¹⁷⁷ plus J591 had internalized more 617-Lu¹⁷⁷ counts than those wells that did not contain J591 (FIG. 2). Furthermore, during the 48- hour observation period, those cells that got both J591 plus 617-Lu¹⁷⁷ retained a greater amount and proportion of their 617-Lu¹⁷⁷ internalized within the cells (FIG. 2). In the case of LNCaP, the internalized counts actually increase between 24 to 48 hours as the cells are taking up 617-Lu¹⁷⁷ that had been shed into the culture medium (FIG. 2). This ‘re-uptake’ phase occurs in LNCaP but not CWR22Rv1. This is due to the greater expression of PSMA receptor on LNCaP than CWR22Rv1 which leads to a greater absolute amount of radioligand having been internalized by LNCaP. This, in turn, leads to a greater absolute amount of radioligand shed by LNCaP than CWR22Rv1 which, in the presence of the higher expression of PSMA is able to re-internalize the ligand.

### Example 3

### Cells That Were Exposed to Both J591 and ACUPA-Cy3 Internalized Approximately 2-fold the ACUPA Dye as Those Cells That Did Not Have J591 Present

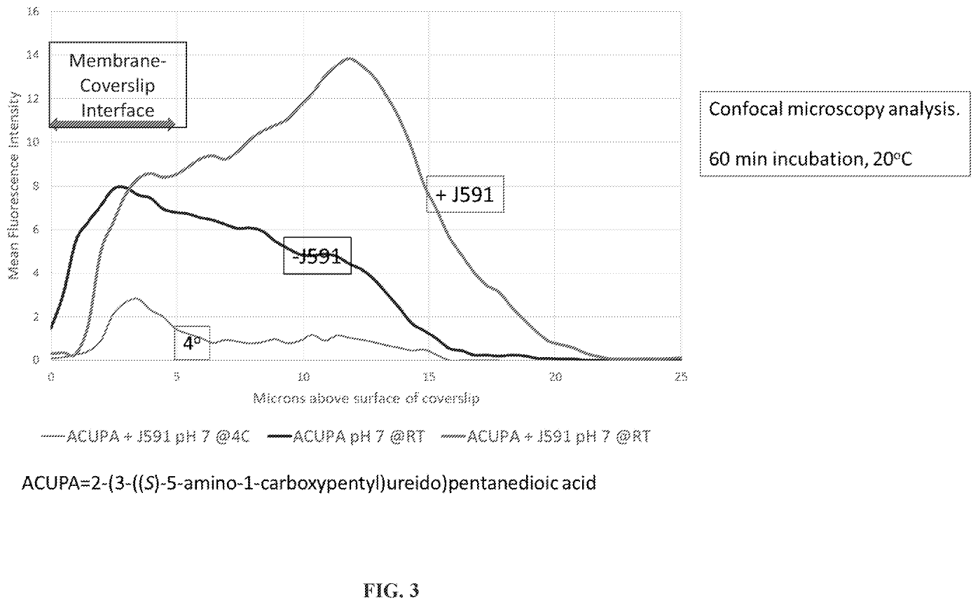

Another technique, confocal microscopy with computer analysis of dye uptake, was also used to compare the relative uptake of the PSMA binding glutamate-urea-lysine [ACUPA (2-(3-((S)-5-amino-1-carboxypentyl) ureido)pentanedioic acid)]-Cy3 in the presence or absence of J591 anti-PSMA antibody. ACUPA-Cy3 was incubated with LNCaP cells in the presence or absence of unlabeled J591. A control condition consisted of ACUPA-Cy3 incubated at 4° C. which prevents internalization and limits ACUPA binding to the cell membrane. The confocal microscope quantitates the dye in a series, or stack, of ‘layers’ from the interface between cell and culture plate to the peak of the cell. The graph shows the dye measurement in mean fluorescence intensity (MFI) at each layer (microns above the plate surface) as the microscope moves from the plane of the plate to the top of the cell (FIG. 3). The area under curve (AUC) of each curve represents the total ACUPA-Cy3 internalized. In this study, cells that were exposed to both J591 and ACUPA-Cy3 internalized approximately 2-fold the ACUPA dye as those cells that did not have J591 present (FIG. 3). At the mid-point of the cell, about 12 microns above the surface of the plate, the presence of the J591 Ab increases the amount of ACUPA-Cy3 by approximately 3-fold. This is consistent with other data that the Ab induces a shift of the ligand from recycling endosome to the lysosomal compartment neighboring the nucleus.

### Example 4

### Effect of “Cold” J591 anti-PSMA Antibody on the Tumor Dosimetry of PSMA-617-Lu¹⁷⁷ in Mouse Xenografts

CWR22rv1 cells (5×10⁶ cells/200 μl matrigel/mouse) were injected into 40 BALB/c nude mice. After tumors were established and growing, all mice were injected intravenously (IV) via tail vein with PSMA-617-Lu¹⁷⁷ (300 μci/per mouse) on day 0. The treatment groups were as follows.

TABLE 2

| Group | PSMA- | | Timing of | Sacrifice |
|---|---|---|---|---|
| (# tumors) | 617-Lu¹⁷⁷ | Combined with: | injection(s) | on: |
| 1 (12) | + | PBS (control) | Combined, day 0 | Day 3 |
| 2 (12) | + | “Cold” Herceptin 25 μg | Combined, day 0 | Day 3 |
| | | (antibody negative control) | | |
| 3 (12) | + | “Cold” J591 25 μg | Combined, day 0 | Day 3 |
| 4 (8) | + | “Cold” J591 50 μg | Combined, day 0 | Day 3 |
| 5 (12) | + | “Cold” J591 25 μg | J591 day −2 (prior | Day 3 |
| | | | to 617-Lu¹⁷⁷) | |
| 6 (12) | + | “Cold” J591 25 μg | J591 day −1 (prior | Day 3 |
| | | | to 617-Lu¹⁷⁷) | |
| 7 (12) | + | “Cold” J591 25 μg | J591 day +1 | Day 3 |
| | | | (after 617-Lu¹⁷⁷) | |

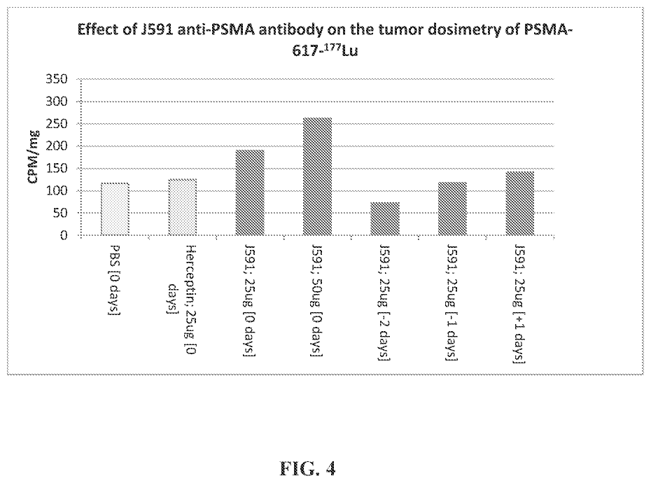

All mice were euthanized 3 days after injection of PSMA-617-Lu^{177.} All tumors were harvested, weighed individually, and radioactivity counted. CPM/mg tumor was calculated and plotted and is shown in FIG. 4.

CWR22Rv1 is a heterogeneous, low PSMA-expressing cell line. The baseline control group (PBS) of tumors measured an average of 150 cpm/mg of tumor at the time of autopsy on day 3 post-injection of PSMA-617-Lu^{177.} Addition of ‘cold’ Herceptin, targeting HER2, an antibody negative control, had no impact on the uptake of 617-Lu^{177.} However, co-administration of cold J591 (to PSMA) at approximately equimolar or 2x molar amount of 617-Lu¹⁷⁷ increased uptake of PSMA-617-Lu¹⁷⁷ by 2-3 fold. When J591 was given 1 day before or 1 day after PSMA-617-Lu^{177,} the enhanced uptake was lost. When J591 was given 2 days before PSMA-617-Lu^{177,} it actually diminished PSMA-617-Lu¹⁷⁷ uptake.

### Example 5

### The Early Impact of J591 anti-PSMA Antibody on PSMA Ligand (PSMA I&T-Lu¹⁷⁷) Measured In Vivo in an Animal Model

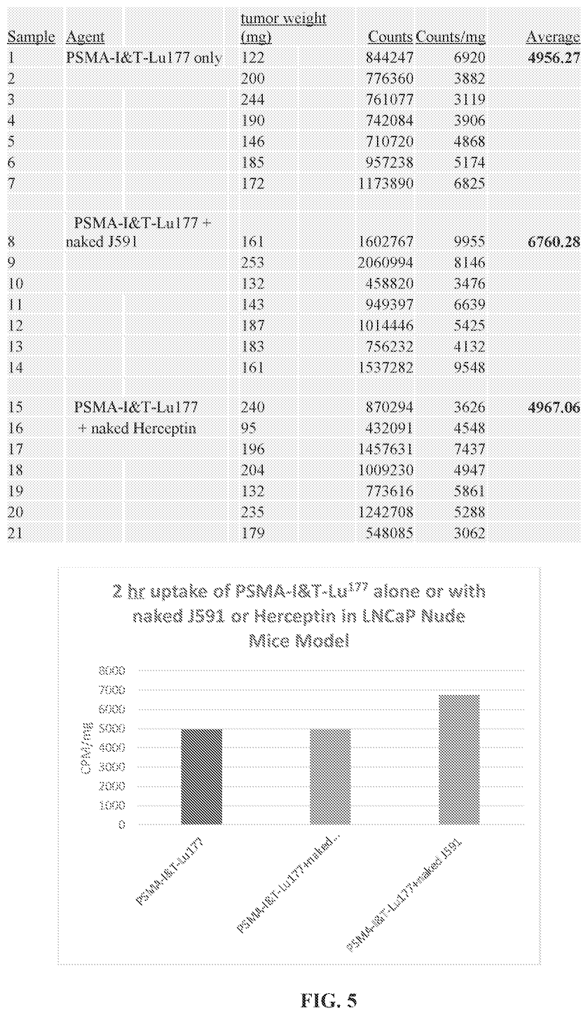

The impact of J591 anti-PSMA antibody on PSMA ligand (PSMA I&T-Lu¹⁷⁷) internalization at an early time point (2 hours) in vivo was measured in vivo in an animal model. In this experiment, there were 3 groups of mice with 7 LNCaP tumors in each group. One group got PSMA I&T-Lu¹⁷⁷ injected IV alone; a 2^{nd} group got PSMA I&T-Lu¹⁷⁷ co-injected with unlabeled Herceptin (anti-Her2) and the 3^{rd} group got PSMA I&T-Lu¹⁷⁷ co-injected with unlabeled J591 (anti-PSMA). To determine the very short-term effect of the combination on tumor uptake of the radiolabeled PSMA I&T, the mice were sacrificed 2 hours after injection, their tumors harvested, weighed and PSMA I&T-Lu¹⁷⁷ radioactivity counted in a gamma counter to determine counts per mg of tumor. The group treated with anti-her2 was a negative control to rule out a non-specific effect of a non-PSMA antibody. The data shows that the anti-Her2 antibody had no effect on the uptake of PSMA I&T-Lu¹⁷⁷ compared to when no antibody was co-injected (FIG. 5). Conversely, the tumors treated with PSMA I&T-Lu¹⁷⁷ plus unlabeled J591 had a mean of 36% higher radioactivity at the 2-hour time point confirming the early effect of the combination treatment (FIG. 5).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating cancer, said method comprising:
providing a first agent comprising a first targeting component coupled to a cancer therapeutic component;
providing a second agent comprising a second targeting component alone, wherein the second agent increases the uptake, internalization, and/or retention of the first targeting component coupled to a cancer therapeutic and
administering, to a subject having cancer, the first and second agents to treat cancer.

2. The method according to claim 1, wherein the first targeting component is selected from the group consisting of a protein, a peptide, and a small molecule, and the second targeting component is an antibody or antigen binding fragment thereof.

3. The method according to claim 1, wherein the first and second targeting components target the same molecular target.

4. The method according to claim 1, wherein the first and second targeting components target different molecular targets on the same cell.

5. The method according to claim 1, wherein the cancer therapeutic component has a maximum tolerated dose, and the maximum tolerated dose of the cancer therapeutic component is given during said administering.

6. The method according to claim 1, wherein the cancer therapeutic component has a maximum tolerated dose, and less than the maximum tolerated dose of the cancer therapeutic component is given during said administering.

7. The method according to claim 1, wherein the cancer therapeutic component is selected from the group consisting of a radionuclide and a chemotherapeutic agent.

8. The method according to claim 7, wherein the cancer therapeutic component is a radionuclide selected from the group consisting of ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁸⁸Rd, ¹⁶⁶Dy, ¹⁶⁶Ho, ¹⁴⁹Pm, ¹⁵¹Pm, ¹⁵³Sm, ¹⁵⁹Gd, ¹⁷²Tm, ¹⁶⁹Yb, ¹⁷⁵Yb, ¹⁷⁷Lu, ¹⁰⁵Rh, ¹¹¹Ag, ¹³¹I, ^{177m}Sn, ²²⁵Ac, ²²⁷Th, ²¹¹At, and combinations thereof.

9. The method according to claim 7, wherein the cancer therapeutic component is a chemotherapeutic agent selected from the group consisting of busulfan, cisplatin, carboplatin, chlorambucil, cyclophosphamide, ifosfamide, dacarbazine (DTIC), mechlorethamine (nitrogen mustard), melphalan, carmustine (BCNU), lomustine (CCNU), 5-fluorouracil (5-FU), capecitabine, methotrexate, gemcitabine, cytarabine (ara-C), fludarabine, dactinomycin, daunorubicin, doxorubicin (Adriamycin), idarubicin, mitoxantrone, paclitaxel, docetaxel, cabazitaxel, etoposide (VP-16), vinblastine, vincristine, vinorelbine, prednisone, dexamethasone, tamoxifen, fulvestrant, anastrozole, letrozole, megestrol acetate, bicalutamide, flutamide, leuprolide, goserelin, L-asparaginase, tretinoin, maytansines, auristatins, pyrrolobenzodiazepines, duocarmycins, and combinations thereof.

10. The method according to claim 1, wherein the cancer is prostate cancer.

11. The method according to claim 10, wherein the first and second targeting components target the prostate-specific membrane antigen (PSMA) receptor.

12. The method according to claim 11, wherein the first targeting component is a PSMA receptor binding peptide or PSMA receptor inhibitor and the second targeting component is a PSMA receptor antibody or an antigen binding portion thereof.

13. The method according to claim 12, wherein the first targeting component is a peptide selected from the group consisting of PSMA-617, PSMA I&T, PSMA I&S, PSMA 1007, PSMA-11, DCFPyL, MIP-1404, MIP-1972, MIP-1095, rh(radiohybrid)PSMA, and other PSMA ligands/inhibitors, while the second targeting component is an antibody selected from the group consisting of J591, J415, E99, J533, D2B, 2G7, and 107-1A4.

14. The method according to claim 12, wherein the first agent is PSMA 617-¹⁷⁷Lu and the second agent is J591.

15. The method according to claim 1, wherein the subject is a human.

16. The method according to claim 1, wherein the cancer is a neuroendocrine cancer.

17. The method according to claim 16, wherein the first and second targeting components target the somatostatin receptor.

18. The method according to claim 17, wherein the first and second targeting components target the somatostatin receptor-2 isoform.

19. The method according to claim 16, wherein the neuroendocrine cancer is selected from the group consisting of carcinoid tumors, gastrinoma, insulinoma, glucagonoma, VIPoma, somatostatinoma, thyroid carcinoma, Merkel cell carcinoma of the skin, tumor of the anterior pituitary, medullary carcinoma, parathyroid tumor, thymus and mediastinal carcinoid tumor, pulmonary neuroendocrine tumor, adrenomedullary tumor, pheochromocytoma, Schwannoma, paraganglioma, and neuroblastoma.

20. The method according to claim 1, wherein the cancer is breast cancer.

21. The method according to claim 20, wherein the first and second targeting components target the HER receptor family.

22. The method according to claim 1, wherein the cancer is non-Hodgkin's Lymphoma.

23. The method according to claim 22, wherein the first and second targeting components target CD20.

24. The method according to claim 1, wherein the first and second agents are different.

25. The method according to claim 1, wherein the first and second targeting components target a cancer cell receptor.

26. A combination therapeutic for treating cancer comprising:
a first agent comprising a first targeting component coupled to a cancer therapeutic and
a second agent comprising a second targeting component alone, wherein the second agent increases the uptake, internalization, and/or retention of the first targeting component coupled to a cancer therapeutic.

27. The combination therapeutic according to claim 26, wherein the first targeting component is selected from the group consisting of a protein, a peptide, and a small molecule, and the second targeting component is an antibody or antigen binding portion thereof.

28. The combination therapeutic according to claim 26, wherein the first and second targeting components target the same molecular target.

29. The combination therapeutic according to claim 26, wherein the first and second targeting components target different molecular targets on the same cell.

30. The combination therapeutic according to claim 26, wherein the cancer therapeutic component is selected from the group consisting of a radionuclide and a chemotherapeutic agent.

31. The combination therapeutic according to claim 30, wherein the cancer therapeutic component is a radionuclide selected from the group consisting of ⁸⁶Re, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁸⁸Rd, ¹⁶⁶Dy, ¹⁶⁶Ho, ¹⁴⁹Pm, ¹⁵¹Pm, ¹⁵³Sm, ¹⁵⁹Gd, ¹⁷²Tm, ¹⁶⁹Yb, ¹⁷⁵Yb, ¹⁷⁷Lu, ¹⁰⁵Rh, ¹¹¹Ag, ¹³¹I, ^{177m}Sn, ²²⁵Ac, ²²⁷Th, ²¹¹At, and combinations thereof.

32. The combination therapeutic according to claim 30, wherein the cancer therapeutic component is a chemotherapeutic agent selected from the group consisting of busulfan, cisplatin, carboplatin, chlorambucil, cyclophosphamide, ifosfamide, dacarbazine (DTIC), mechlorethamine (nitrogen mustard), melphalan, carmustine (BCNU), lomustine (CCNU), 5-fluorouracil (5-FU), capecitabine, methotrexate, gemcitabine, cytarabine (ara-C), fludarabine, dactinomycin, daunorubicin, doxorubicin (Adriamycin), idarubicin, mitoxantrone, paclitaxel, docetaxel, cabazitaxel, etoposide (VP-16), vinblastine, vincristine, vinorelbine, prednisone, dexamethasone, tamoxifen, fulvestrant, anastrozole, letrozole, megestrol acetate, bicalutamide, flutamide, leuprolide, goserelin, L-asparaginase, tretinoin, maytansines, auristatins, pyrrolobenzodiazepines, duocarmycins, and combinations thereof.

33. The combination therapeutic according to claim 26, wherein the cancer is prostate cancer.

34. The combination therapeutic according to claim 33, wherein the first and second targeting components target the prostate-specific membrane antigen (PSMA) receptor.

35. The combination therapeutic according to claim 34, wherein the first targeting component is a PSMA receptor binding peptide or PSMA receptor inhibitor and the second targeting component is a PSMA receptor antibody or an antigen binding portion thereof.

36. The combination therapeutic according to claim 35, wherein the first targeting component is a peptide selected from the group consisting of PSMA-617, PSMA I&T, PSMA I&S, PSMA 1007, PSMA-11, DCFPyL, MIP-1404, MIP-1972, MIP-1095, rh(radiohybrid)PSMA, and other PSMA ligands/inhibitors, while the second targeting component is an antibody selected from the group consisting of J591, J415, E99, J533, D2B, 2G7, and 107-1A4.

37. The combination therapeutic according to claim 35, wherein the first agent is PSMA 617-¹⁷⁷Lu or PSMA I&T-¹⁷⁷Lu and the second agent is J591.

38. The combination therapeutic according to claim 26, wherein the cancer is a neuroendocrine cancer.

39. The combination therapeutic according to claim 38, wherein the first and second agents target the somatostatin receptor.

40. The combination therapeutic according to claim 39, wherein the first and second targeting components target the somatostatin receptor-2 isoform.

41. The combination therapeutic according to claim 38, wherein the neuroendocrine cancer is selected from the group consisting of carcinoid tumors, gastrinoma, insulinoma, glucagonoma, VIPoma, somatostatinoma, thyroid carcinoma, Merkel cell carcinoma of the skin, tumor of the anterior pituitary, medullary carcinoma, parathyroid tumor, thymus and mediastinal carcinoid tumor, pulmonary neuroendocrine tumor, adrenomedullary tumor, pheochromocytoma, Schwannoma, paraganglioma, and neuroblastoma.

42. The combination therapeutic according to claim 26, wherein the cancer is breast cancer.

43. The combination therapeutic according to claim 42, wherein the first and second targeting components target the HER receptor family.

44. The combination therapeutic according to claim 26, wherein the cancer is non-Hodgkin's Lymphoma.

45. The combination therapeutic according to claim 44, wherein the first and second targeting components target CD20.

46. The combination therapeutic according to claim 26, wherein the first and second targeting components are different.

47. The combination therapeutic according to claim 26, wherein the first and second targeting components target a cancer cell receptor.

48. A method of imaging cancer in a subject, said method comprising:
providing a first agent comprising a first targeting component coupled to an imaging component;
providing a second agent comprising a second targeting component alone, wherein the second agent increases the uptake, internalization, and/or retention of the first targeting component coupled to an imaging component and
administering, to a subject having cancer, the first and second agents to image cancer.

49. The method according to claim 48, wherein the first targeting component is selected from the group consisting of a protein, a peptide, and a small molecules, and the second targeting component is an antibody or binding fragment thereof.

50. The method according to claim 48, wherein the first and second targeting components target the prostate specific membrane antigen (PSMA) receptor.

51. The method according to claim 50, wherein the first targeting component is a PSMA receptor binding peptide or PSMA receptor inhibitor and the second targeting component is a PSMA receptor antibody or an antigen binding portion thereof.

52. The method according to claim 51, wherein the first targeting component is a peptide selected from the group consisting of PSMA-617, PSMA I&T, PSMA I&S, PSMA 1007, PSMA-11, DCFPyL, MIP-1404, MIP-1972, MIP-1095, rh(radiohybrid)PSMA, and other PSMA ligands/inhibitors, while the second targeting component is an antibody selected from the group consisting of J591, J415, E99, J533, D2B, 2G7, and 107-1A4.

53. The method according to claim 48, wherein the cancer is a neuroendocrine cancer.

54. The method according to claim 53, wherein the first and second targeting components target the somatostatin receptor.

55. The method according to claim 54, wherein the first and second targeting components target the somatostatin receptor-2 isoform.

56. The method according to claim 48, wherein the cancer is breast cancer.

57. The method according to claim 56, wherein the first and second targeting components target the HER receptor family.

58. The method according to claim 48, wherein the cancer is non-Hodgkin's Lymphoma.

59. The method according to claim 58, wherein the first and second targeting components target CD20.

60. The method according to claim 48, wherein the imaging component is selected from the group consisting of radiolabels, fluorescent labels, nuclear magnetic resonance active labels, chemiluminescent labels, enzymatic markers, radiation emitters, and combinations thereof.

61. A combination imaging system for imaging cancer comprising:
a first agent comprising a first targeting component coupled to an imaging component and
a second agent comprising a second targeting component alone, wherein the second agent increases the uptake, internalization, and/or retention of the first targeting component coupled to an imaging component.

62. The combination imaging system according to claim 61, wherein the first targeting component is selected from the group consisting of a protein, a peptide, and small molecule, and the second targeting component is an antibody or antigen binding portion thereof.

63. The combination imaging system according to claim 61, wherein the first and second targeting components target the prostate specific membrane antigen (PSMA) receptor.

64. The combination imaging system according to claim 61, wherein the first targeting component is a PSMA receptor binding peptide or PSMA receptor inhibitor and the second targeting component is a PSMA receptor antibody or antigen binding portion thereof.

65. The combination imaging system according to claim 61, wherein the first targeting component is a peptide selected from the group consisting of PSMA-617, PSMA I&T, PSMA I&S, PSMA 1007, PSMA-11, DCFPyL, MIP-1404, MIP-1972, MIP-1095, rh(radiohybrid)PSMA, and other PSMA ligands/inhibitors, while the second targeting component is an antibody selected from the group consisting of J591, J415, E99, J533, D2B, 2G7, and 107-1A4.

66. The combination imaging system according to claim 61, wherein the cancer is a neuroendocrine cancer.

67. The combination imaging system according to claim 60, wherein the first and second targeting components target the somatostatin receptor.

68. The combination imaging system according to claim 61, wherein the first and second targeting components target the somatostatin receptor-2 isoform.

69. The combination imaging system according to claim 61, wherein the cancer is breast cancer.

70. The combination imaging system according to claim 61, wherein the first and second targeting components target the HER receptor family.

71. The combination imaging system according to claim 61, wherein the cancer is non-Hodgkin's Lymphoma.

72. The combination imaging system according to claim 61, wherein the first and second targeting components target CD20.

73. The combination imaging system according to claim 61, wherein the imaging component is selected from the group consisting of radiolabels, fluorescent labels, nuclear magnetic resonance active labels, chemiluminescent labels, enzymatic markers, radiation emitters, and combinations thereof.
